(19) United States
(12) Patent Application Publication
(10) Pub. No.: US 2024/0010612 A1
(43) Pub. Date: Jan. 11, 2024

(54) THIOL FUNCTIONALIZED CONDUCTOR COMPOUND AND METHOD FOR MAKING SAME

(71) Applicants: BLUE SOLUTIONS CANADA INC., Boucherville (CA); CAPACITOR SCIENCES INCORPORATED, Menlo Park, CA (US)

(72) Inventors: Qiujie ZHAO, Menlo Park, CA (US); Siwei LIANG, Menlo Park, CA (US); Jin YANG, Menlo Park, CA (US); Sarah DEGRAS, BRossard (CA); Patrick LEBLANC, Boucherville (CA)

(21) Appl. No.: 18/339,684

(22) Filed: Jun. 22, 2023

Related U.S. Application Data
(60) Provisional application No. 63/356,176, filed on Jun. 28, 2022.

(51) Int. Cl. C07C 311/48; H01M 10/0565; H01M 4/137

(52) U.S. Cl. CPC C07C 311/48; H01M 10/0565; H01M 4/137; H01M 2300/0082

(57) ABSTRACT

The present technology relates to thiol functionalized conductors which can be grafted onto polymers and methods for making same. In certain embodiments, the thiol functionalized conductors can be grafted onto polymers with low Tg to synthesize single-ion conducting polymer electrolytes (SIPE) having improved conductivity. The thiol functionalized conductor compound comprises a covalently attached sulfonimide anion on one end, which is associated with a monovalent cation; 1-3 hydrocarbon chains as the linker (L); 0-2 functional groups (R); and a thiol group on the other end of the compound.

### CROSS-REFERENCE TO RELATED APPLICATION

The present patent application claims the rights and benefits to U.S. Provisional Application No. 63/356,176, filed on Jun. 28, 2022, the content of which is incorporated herein by reference.

### TECHNICAL FIELD

The present technology generally relates to single-ion conducting polymer electrolytes, and in particular to thiol functionalized conductor compounds and methods for making same.

### BACKGROUND

A lithium battery using a lithium metal as a negative electrode has excellent energy density. However, with repeated cycles, such a battery can be subject to dendrite growth on the surface of the lithium metal electrode when recharging the battery, as the lithium ions are unevenly re-plated on the surface of the lithium metal electrode. To minimize the effect of the morphological evolution of the surface of the lithium metal anode including dendrite growth, a lithium metal battery typically uses a pressure system and a solid polymer electrolyte adapted to resist the pressure applied thereto as described in U.S. Pat. No. 6,007,935 (incorporated herein by reference). Over numerous cycles, dendrites on the surface of the lithium metal anode, however, may still grow to penetrate the solid polymer electrolyte, and eventually cause ‘soft’ short circuits between the negative electrode and the positive electrode, resulting in decreasing or poor performance of the battery. Therefore, the growth of dendrites may still deteriorate the cycling characteristics of the battery and constitutes a major limitation with respect to the optimization of the performance of lithium batteries having a metallic lithium anode.

Various types of solid polymer electrolytes adapted for use with lithium metal electrodes have been developed since the late 1970s to overcome this issue but have been found to lack in conductivity and/or mechanical properties. Single-ion conducting polymer electrolytes (SIPE) have, however, emerged as promising candidates, as the transference number of lithium cation approaches unity, and therefore prevents the formation of concentration gradients across the electrolyte, and dendrite formation as a result.

Currently, existing routes of synthesis of single-ion polymer electrolytes include synthesis of poly(ethylene oxide) methacrylate lithium sulfonyl(trifluoromethylsulfonyl)imide) (PEOMA-TFSI-Li+) monomers via a copper-catalyzed alkyne-azide “click chemistry” cycloaddition as illustrated in FIG. 1. The synthesis of azide-clicked PEOMA-TFSI-Li⁺ is however complex and not suitable for scale-up production for at least the following reasons: (1) azide and alkyne functional groups must be installed on the precursors; (2) the intermediate molecules are expensive to make and not commercially available; (3) alkali azide salts, such as LiN₃ and NaN₃, are dangerous and hard to handle in large quantity, preventing the scale up of the precursors; (4) the final polymer is a PEO based polymer, which is not suitable for high voltage applications; and (5) the chemistry is not versatile in terms of functional group availability as commercial polymers with internal triple bonds are rare.

The inventors of the present technology have however recently discovered novel routes of synthesis of single-ion polymer electrolytes which include the synthesis of LiTFSI monomers with reduced cost and high atom economy (i.e., produce minimal reactant waste) by virtue of, inter alia, comprising a single step and bypassing the synthesis of nitrogen-based organic cation intermediates. These novel synthesis routes can be applied to make SIPEs with styrene, acrylate or methacrylate backbones. However, the resulting homopolymers derived from such LiTFSI monomers have high glass transition temperatures (Tg) and low conductivity at room temperature which hinder their performance as electrolytes and ultimately the performance of the battery. Moreover, such LiTFSI monomers do not comprise functional groups suitable for grafting onto polymers with low Tgs to help alleviate those defects.

Therefore, there is a need for alternative or improved methods of synthesis of SIPES which comprise various polymer backbones with low Tg and overcome or reduce at least some of the above-described problems.

### SUMMARY

From a broad aspect, the present technology relates to thiol functionalized conductors. In certain embodiments, the thiol functionalized conductors can be grafted onto polymers having low Tg to synthesize single-ion conducting polymer electrolytes (SIPE) with improved conductivity.

From one aspect there is provided a thiol functionalized conductor compound having formula I:

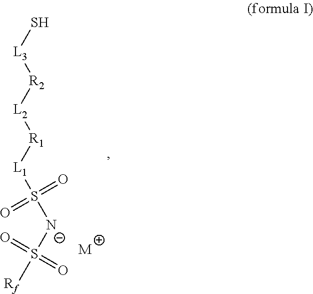

wherein:
R_{f} is F, CF₃, CF₂CF₃, (CF₂)ₙCF₃, wherein n is ≥1, C₆F₅, a branched C₃-C₄ fluoroalkyl group, —(CF₂CF₂O)ₘ—CF₂CF₃ wherein m=1, 2 or 3, —(CF₂O)ₚ/(CF₂CF₂O)_{q}—CF₂CF₃ wherein 1≤p≤10, 1≤q≤10, and the (CF₂O) and (CF₂CF₂O) units are randomly copolymerized or an aryl substituted with at least one fluorine and at least one electron-withdrawing group;
M⁺ is a monovalent cation;
L₁ is a n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
L₂ is absent or present and when present is a n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
L₃ is absent or present and when present is n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
R₁ is an ether, a thioether, an ester, an amide, a urethane, urea, a secondary amine, or a tertiary amine; and
R₂ is absent or present and when present is an ether, a thioether, an ester, an amide, a urethane, urea, a secondary amine, or a tertiary amine.

From another aspect, there is provided a method for the synthesis of the thiol functionalized conductor compounds of the present technology, the method comprising reacting a thiol compound with a lithium bis(trifluoromethanesulfonyl)imide (LiTFSI)-monomer having a C═C in its backbone.

From another aspect, there is provided a single-ion conducting polymer electrolyte comprising the thiol functionalized conductor compound of the present technology.

From another aspect, there is provided a solid-state battery comprising a positive electrode, a negative electrode and a single-ion conducting polymer electrolyte comprising the thiol functionalized conductor compound of the present technology.

From another aspect, the thiol functionalized conductor compound of the present technology can be grafted onto different polymers.

From another aspect, the methods for the synthesis of the thiol functionalized conductor compound of the present technology are effective with thiol compounds such as 1,3-propane-dithiol and 2,2′-(Ethylenedioxy)diethanethiol which are cheap and therefore provide an economical route of synthesis for single-ion polymer electrolytes.

From another aspect, the methods of the present technology are facile and do not require heating.

### BRIEF DESCRIPTION OF THE DRAWINGS

Reference will now be made to the accompanying drawings.

FIG. 1 illustrates an existing synthesis route for poly(ethylene oxide) methacrylate lithium sulfonyl(trifluoromethylsulfonyl)imide) (PEOMA-TFSI-Li+) according to Sipei Li et al., ACS Energy Lett. 2018, 3, 1, 20-27 (incorporated herein by reference) using an azide-alkyne click chemistry.

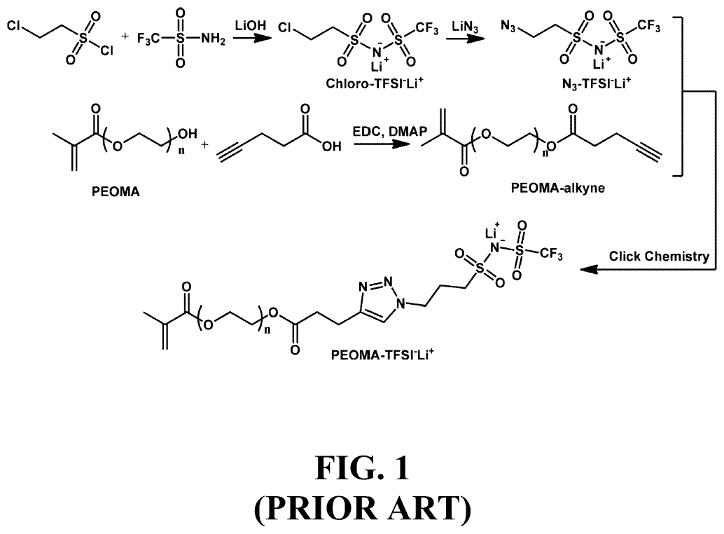

FIG. 2 illustrates an ¹H-NMR spectra of a thiol functionalized conductor compound according to one embodiment of the present technology (bottom panel) compared with the starting materials: a LiTFSI-acrylate compound (top panel), and 1,3-propanedithiol (middle panel).

FIG. 3 illustrates an ¹H-NMR integration of the thiol functionalized conductor compound of FIG. 2 in which mono-substitution of dithiol is evident by: (1) 1:1 ratio of peak b′ vs. f1 and (2) existence of peak f2 and g′.

FIG. 4 is a schematic representation of a plurality of electrochemical cells forming a solid-state battery comprising the single-ion conducting polymer electrolytes of the present technology.

### DETAILED DESCRIPTION

### Definition

The use of “including”, “comprising”, or “having”, “containing”, “involving” and variations thereof herein, is meant to encompass the items listed thereafter as well as, optionally, additional items.

It must be noted that, as used in this specification and the appended claims, the singular form “a”, “an” and “the” include plural referents unless the context clearly dictates otherwise.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each is set out individually herein.

As used herein, the expression “single-ion conducting polymer” is a polymer comprising an immobile anion as part of its chemical structure. As used herein, the expression immobile anion refers to anions which are not displaced during the charge/discharge cycles of the battery.

As used herein the term “facile” refers to a chemical reaction which takes place readily.

As used herein, the term “work-up” refers to a series of manipulations required to isolate and purify the product of a chemical reaction.

As used herein, the term “substantially” means to a great or significant extent.

As used herein, the expressions “click chemistry” or “click reaction” refers to a reaction which is simple; has a high efficiency, a high yield; and generates byproducts which are stereospecific and can be easily removed. Moreover, such reactions can be conducted in easily removable or benign solvents. Click chemistry was conceptualized by Sharpless et al., Angew. Chem. Int. Ed. 2001, 40, 2004-2021, incorporated herein by reference.

As used herein, the term “about” in the context of a given value or range refers to a value or range that is within 20%, preferably within 10%, and more preferably within 5% of the given value or range.

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

Broadly, the present technology relates to thiol functionalized conductors which can be grafted onto polymers, such as commercially available polymers, including, but not limited to, polyethylene oxide (PEO), polyvinylidene fluoride (PVDF), and poly(acrylonitrile-co-butadiene). In some instances, the grafting reaction of the thiol functionalized conductors with such polymers comprises a thiol-ene “click” reaction which generates single-ion conducting polymer electrolytes (SIPE) having low Tg. As such, the SIPE comprising the thiol functionalized conductors of the present technology have higher conductivity than the SIPEs obtained by the polymerization of LiTFSI-containing monomers having styrene, acrylate or methacrylate backbones.

In certain embodiments, the thiol functionalized conductor compounds of the present technology comprise a covalently attached sulfonimide anion on one end, which is associated with a monovalent cation; 1-3 hydrocarbon chains as linkers (L); 1-2 functional groups (R); and a thiol group on the other end of the compound. In some embodiments, the thiol functionalized conductor compound has formula I:

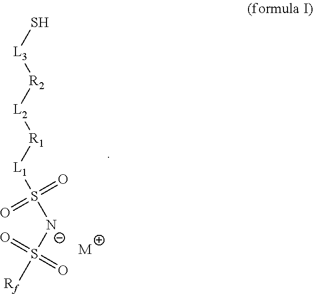

In certain embodiments, R_{f} is F, CF₃, CF₂CF₃, (CF₂)ₙCF₃ wherein n is ≥1, C₆F₅, a branched C₃-C₄ fluoroalkyl group, a linear perfluorethylether group, such as —(CF₂CF₂O)ₘ—CF₂CF₃ wherein m=1, 2 or 3, —(CF₂O)ₚ/(CF₂CF₂O)_{q}—CF₂CF₃ wherein 1≤p≤10, 1≤q≤10, and the (CF₂O) and (CF₂CF₂O) units are randomly copolymerized, or an aryl substituted with at least one fluorine and at least one electron-withdrawing group. In some embodiments, the branched C₃-C₄ fluoroalkyl group comprises —CF—(CF₃)₂, —CF(CF₃)—CF₂—CF₃, and CF₂—CF—(CF₃)₂. In other embodiments, the electron withdrawing group is selected from —CN, —NO₂, —CF₃, and —SO₂CF₃. In further embodiments, the aryl compound substituted with the at least one fluorine and the at least one electron-withdrawing group is —C₆F₄—CF₃, or —C₆F₄—SO₂CF₃. In yet further embodiments, R_{f} is CF₃.

In certain embodiments, M⁺ in the thiol functionalized conductor compound is a monovalent cation. In some embodiments, the monovalent cation is an alkali metal cation. In other embodiments, the alkali metal cation is H⁺, K⁺, Na⁺, Li⁺, Rb⁺, or Cs⁺. In yet other embodiments, the alkali metal cation is Li⁺.

L₁, L₂, and L₃ in the thiol functionalized conductor compound are linkers, which at least connect the sulfonimide anion on one end with the thiol group on the other end of the compound.

In certain embodiments, L₁, is an-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group. In some embodiments, L₁ is a n-alkyl group and n is 1, 2, 3, 4, 5 or 6. In other embodiments, L₁ is (CH₂)₃.

L₂ may be either absent or present. When present, L₂ is a n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group. In some embodiments, L₂ is a n-alkyl group and n is 1, 2, 3, 4, 5 or 6. In other embodiments, L₂ is (CH₂)₂.

L₃ may also be either absent or present. When present, L₃ is n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group. In some embodiments, L₃ is a n-alkyl group and n is 2, 3, 4, 5 or 6. In other embodiments, L₃ is (CH₂)₃.

R₁ and R₂ in the thiol functionalized conductor compound are functional groups. R₂ may either be absent or present. R₁ and R₂ (when present) are each independently an ether, a thioether, an ester, an amide, a urethane, urea, a secondary amine, or a tertiary amine. In some embodiments, R₁ is an ester. In other embodiments, R₂ is a thioether.

In one embodiment, R_{f} is CF₃, M⁺ is Li⁺, L₁, L₂, and L₃ are each an n-alkyl group wherein n is 2, 3, 4, 5 or 6, R₁ is an ester, and R₂ is a thioether. In another embodiment, R_{f} is CF₃, M⁺ is Li⁺, L₁ and L₃ are (CH₂)₃, L₂ is (CH₂)₂, R₁ is an ester, and R₂ is a thioether.

In certain embodiments, the thiol functionalized conductor compound of the present technology has formula II or formula III:

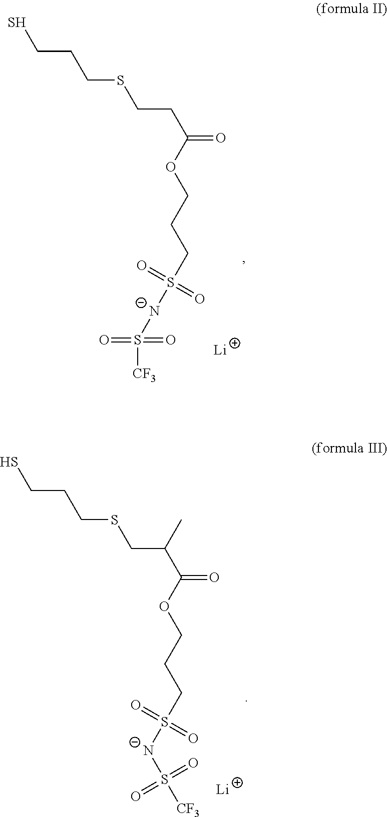 [SMILES: O=C(CCSCCCS)OCCCS(=O)(=O)[N-]S(=O)(=O)C(F)(F)F ; [Li+] ; CC(CSCCCS)C(=O)OCCCS(=O)(=O)[N-]S(=O)(=O)C(F)(F)F ; [Li+]]

From another aspect, the present technology relates to methods of synthesis of the thiol functionalized conductor compound disclosed herein. In certain embodiments, the methods of the present technology comprise reacting a thiol compound with a lithium bis(trifluoromethanesulfonyl)imide (LiTFSI)-monomer having a C═C in its backbone. Advantageously, the methods of the present technology comprise a single step. Moreover, direct addition of thiol on the LiTFSI-monomers in the methods of the present technology provide an easier route of synthesis than thiol functionalized LiTFSI-monomers with the thiol directly linked to the LiTFSI via an alkyl chain.

In certain embodiments, the reaction of the thiol compound with the LiTFSI-monomer having the C═C in its backbone is a thiol-ene “click” reaction. Advantageously, thiol-ene click reactions are simple and highly efficient. Such reactions also allow the creation of a large variety of new polymer structures while enabling great spatial and temporal control of the materials.

In certain embodiments, the C═C in the LiTFSI-monomer is at one end of the molecule. In some embodiments, the C═C is at one end of the molecule and the sulfonimide anion is at the other (opposite) end of the LiTFSI-monomer. In other embodiments, the LiTFSI-monomer has the following formula IV, formula V, formula VI, formula VII, or formula VIII:

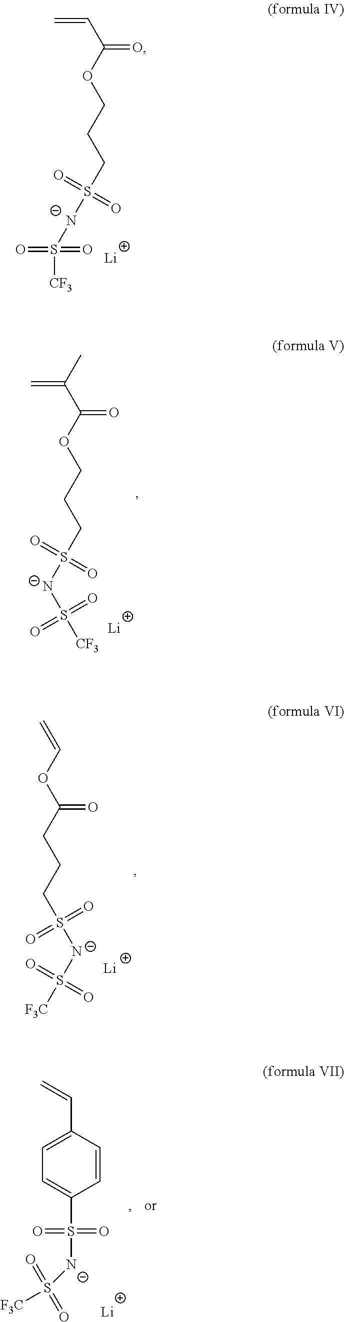

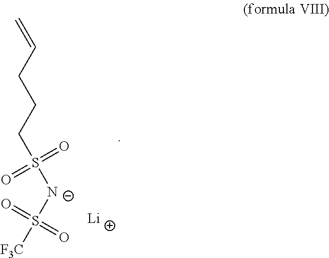 [SMILES: C=CCCCS(=O)(=O)[N-]S(=O)(=O)C(F)(F)F ; [Li+]]

In certain embodiments, the thiol compound is an n-alkyl dithiol, an ethylene glycol based dithiol, a PEO-based dithiol, 2,2′-Thiodiethanethiol, 2,3-Dimercapto-1-propanol, 1,2-benzene-dithiol, 1,3-benzene-dithiol, 1,4-benzene-dithiol, 1,4,benzenedimethanethiol, Toluene-3, 4-dithiol, Biphenyl-4,4′-dithiol, p-Terphenyl-4,4′-dithiol, 1,3-propane-dithiol, or 2,2′-(Ethylenedioxy)diethanethiol. In some embodiments, the n-alkyl dithiol has the formula (SH—(CH₂)ᵣ—SH, wherein r=2, 3, 4, 5, 6, 8, 9, 11, or 16. In other embodiments, the ethylene glycol based dithiol has the formula (SH—(CH₂CH₂O)ₛ—CH₂CH₂—SH, wherein s=2, 3, or 5. In yet other embodiments, the PEO-based dithiol has the formula SH-PEO-SH, wherein the PEO has a number average molecular weight (Mn) of about 1000, about 1500, about 3400, or about 8000. In further embodiments, the thiol compound is 1,3-propane-dithiol. In yet further embodiments, the thiol compound is 2,2′-(Ethylenedioxy)diethanethiol. Advantageously, 1,3-propane-dithiol, and 2,2′-(Ethylenedioxy)diethanethiol are the cheapest dithiols available on the market which provide for an economical way of synthesizing SIPE.

In some embodiments, the method comprises reacting an excess amount of the thiol compound with the LiTFSI-monomer. In other embodiment, the method comprises reacting about 1 to about 4 equivalent of the thiol compound with the LiTFSI-monomer. In yet other embodiments, the method comprises reacting about 1 to about 2 equivalent, about 1 to about 3 equivalent, about 1.5 to about 2.5 equivalent, about 1 to about 1.5 equivalent, about 2 equivalent, or about 1.3 equivalent of the thiol compound with the LiTFSI-monomer.

In certain embodiments, the methods of the present technology comprise reacting the thiol compound and the LiTFSI-monomer in bulk (i.e., without solvent). Such embodiments are plausible when the reactants are miscible in one another. In other embodiments, the methods of the present technology comprise reacting the thiol compound and the LiTFSI-monomer in a solvent. In some embodiments, the solvent is water, methanol, ethanol, isopropanol, anhydrous methyl cyanide (MeCN), tetrahydrofuran (THF), acetone, dimethylformamide (DMF), or dimethyl sulfoxide (DMSO). In some embodiments, the thiol compound and the LiTFSI-monomer are reacted in THF.

In certain embodiments, reacting the thiol compound and the LiTFSI-monomer comprises dissolving the thiol compound and the LiTFSI-monomer together in a solvent. In other embodiments, reacting the thiol compound and the LiTFSI-monomer comprises dissolving the thiol compound in a first solvent, dissolving the LiTFSI-monomer in a second solvent and adding the dissolved LiTFSI-monomer in the second solvent to the thiol compound dissolved in the first solvent. In some embodiments, the first solvent and the second solvent are the same solvent. In other embodiments, the first solvent and the second solvent are different solvents. In such embodiments, the two different solvents are miscible in one another. The first solvent and second solvent may be any of the solvents disclosed above.

In certain embodiments, the LiTFSI-monomer dissolved in the second solvent is added to the thiol compound dissolved in the first solvent slowly and/or and in dropwise fashion. This prevents temperature jumps and solvent evaporation. The methods of the present technology, however, are not limited to a particular order in which the reagents are added. Therefore, it is understood that in other embodiments, the thiol compound dissolved in the first solvent may be added to the LiTFSI-monomer dissolved in the second solvent, for example. In certain implementations of the latter embodiments, the thiol compound dissolved in the first solvent may be added to the LiTFSI-monomer dissolved in the second solvent slowly and/or in a dropwise fashion.

In certain embodiments, the methods of the present technology do not require a heating step. Specifically, in certain embodiments, the methods of the present technology comprise reacting the thiol compound and the LiTFSI-monomer at a temperature of between about 15° C. and about 30° C. In other embodiments, the method comprises reacting the thiol compound and the LiTFSI-monomer at a temperature of about 15° C., about 20° C., about 25° C. (room temperature (RT)), or about 30° C. In yet other embodiments, the method comprises reacting the thiol compound and the LiTFSI-monomer at a temperature of about 25° C. (RT).

In other embodiments, the methods of the present technology comprise reacting the thiol compound and the LiTFSI-monomer for about 12 hours to about 24 hours. In further embodiments, the methods of the present technology comprise reacting the thiol compound and the LiTFSI-monomer for about 14 hours to about 22 hours, about 16 hours to about 20 hours, or about 18 hours. In further embodiments, the methods of the present technology comprise reacting the thiol compound and the LiTFSI-monomer for at least about 12 hours.

In further embodiments, the method of the present technology comprises adding a catalyst to the reaction of the thiol compound and the LiTFSI-monomer. As used herein, the term “catalyst” refers to a substance that can be added to a reaction to increase the reaction rate without getting consumed in the process. In certain embodiments, the catalyst is triethylamine (Et₃N), diethylamine, di-n-propylamine, a C₂-C₆ primary amine, N,N,N′,N′-Tetramethyl-1,8-naphthalenediamine, 1,8-diazabicyclo[5.4.0]undec-7-ene (DBU), 1,5-diazabicyclo[4.3.0]non-5-ene (DBN), tripropylphosphine, dimethylphenylphosphine diphenylmethylphosphine, or triphenylphosphine. In some embodiments, the catalyst is Et₃N. In other embodiments, the catalyst is added at an amount of between about 0.05 mol % and about 30 mol %.

In further embodiments, the methods of the present technology comprise adding a free radical initiator to the reaction of the thiol compound and the LiTFSI-monomer. As used herein, the expression “free radical initiator” refers to substances that can produce radical species under mild conditions and promote radical reactions. In the methods of the present technology, free radical initiators may be used to generate a thiyl radical from the thiol compound and/or to complete the reaction between the thiol compound and the LiTFSI-monomer. In some embodiments, the free radical initiator is a thermal activated free radical initiator. In other embodiments, the free radical initiator is a photochemically activated free radical initiator. In further embodiments, the free radical initiator is Azobisisobutyronitrile (AIBN), Benzyl peroxide, 4,4′-Azobis(4-cyanovaleric acid) (ACVA), 2,2-Dimethoxy-2-phenylacetophenone (DMPA, Irgacure 651), 2-Hydroxy-2-methylpropiophenone (Irgacure 1173), or 2-Hydroxy-4′-(2-hydroxyethoxy)-2-methylpropiophenone (Irgacure 2959).

In further embodiments, the free radical initiator is added at an amount of between about 0.05 mol % and about 5 mol %, between about 0.1 mol % and about 2 mol %, or between about 0.5 mol % and about 1 mol %.

In certain embodiments, the free radical initiator is photochemically activated by UV light. In some embodiments, the UV light has a wavelength of between about 250 nm and about 450 nm, between about 300 nm and about 400 nm or about 365 nm. In further embodiments, the free radical initiator is photochemically activated for a period of between about 1 minute to about 2 hours, between about 5 minutes to about 1 hour, between about 10 minutes to about 40 minutes, between about 10 minutes and about 50 minutes, between about 15 minutes and about 45 minutes, or about 30 minutes. In yet further embodiments, the free radical initiator is DMPA added at an amount of between about 0.5 mol % and about 1 mol % and irradiated with UV light for a duration of between about 10 minutes to about 40 minutes. In other embodiments, the free radical initiator is DMPA added at an amount of between about 0.5 mol % and about 1 mol %, irradiated with UV light having a wavelength of about 365 nm for a duration of about 30 minutes.

In certain embodiments, any one or more of the catalyst or the free radical initiator, or a combination thereof, may be added to the reaction of the thiol compound and the LiTFSI-monomer at the step of dissolving the thiol compound in the first solvent, dissolving the LiTFSI-monomer in the second solvent, both at the steps of dissolving the thiol compound in the first solvent and dissolving the LiTFSI-monomer in the second solvent, or at the step of dissolving the thiol compound and the LiTFSI-monomer together in a solvent. In some embodiments, the catalyst is added at the step of dissolving the thiol compound in the first solvent. In other embodiments, the free radical initiator is added at the step of dissolving the thiol compound and the LiTFSI-monomer together in a solvent.

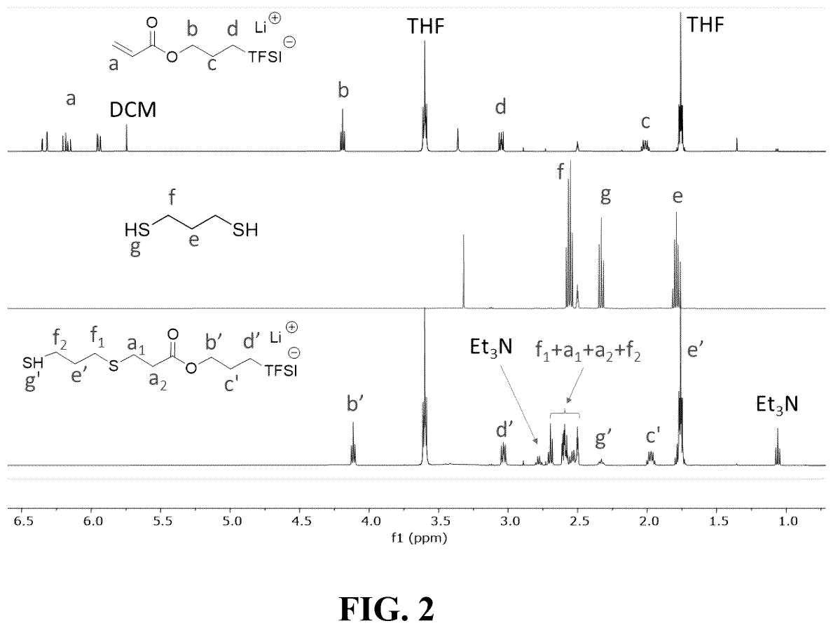

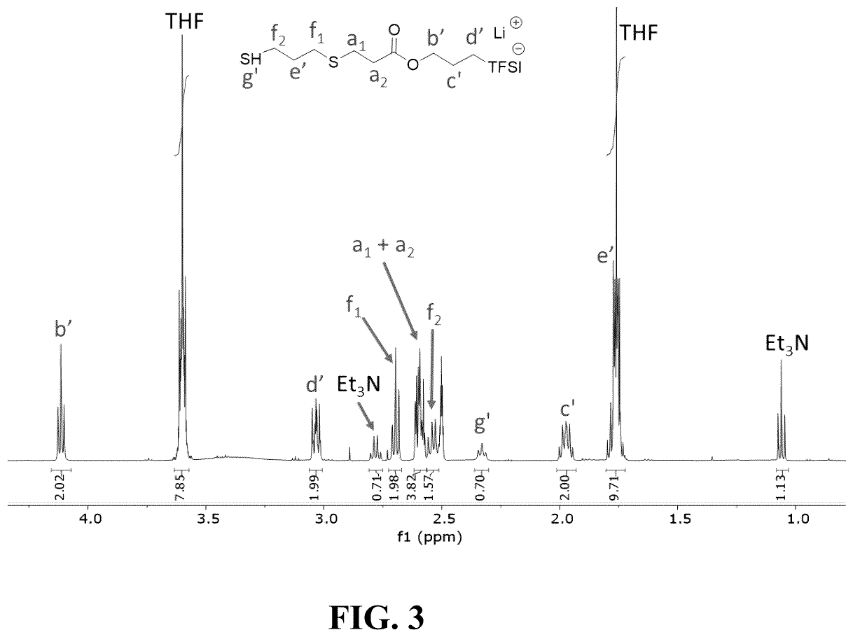

Advantageously, the methods of the present technology yield a mono-substituted thiol functionalized conductor compound as their major product as confirmed by ¹H-NMR Spectra (FIGS. 2 and 3). This product may be easily worked-up to purify and isolate same. Therefore, in certain embodiments, the methods of the present technology further comprise precipitating the thiol functionalized conductor compound. In certain embodiments, precipitation of the thiol functionalized conductor compound is performed in hexane, pentane, cyclohexane, octane, or dietheyl ether. Advantageously, the thiol compound used in the methods of the present technology is soluble in such solvents, thereby allowing for the excess thiol compound to be substantially removed in the precipitating step.

In certain embodiments, the mass yield of the thiol functionalized conductor compound obtained by the methods of the present technology is between about 60% and about 99%, between about 70% and about 90%, between about 80% and about 90%, about 80%, or about 85%.

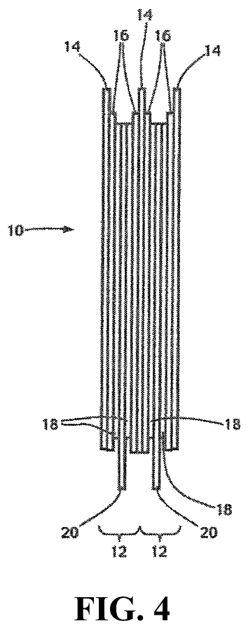

From another aspect, the present technology relates to solid-state batteries having a plurality of electrochemical cells, each electrochemical cell comprising a positive electrode, a negative electrode, and an electrolyte layer disposed therebetween. FIG. 4 schematically illustrates a solid-state battery 10 having a plurality of electrochemical cells 12 each including an anode or negative electrode film 14, a solid electrolyte 16, and a cathode or positive electrode film 18 layered onto a current collector 20. The solid electrolyte 16 typically includes a lithium salt to provide ionic conduction between the anode 14 and the cathode 18. In certain embodiments, the anode film 14 is made of a sheet of metallic lithium having a thickness ranging from about 20 microns to about 100 microns. In other embodiments, the solid electrolyte 16 has a thickness ranging from about 5 microns to about 50 microns. In further embodiments, the positive electrode film 18 has a thickness ranging from about 20 microns to about 100 microns. The thiol functionalized conductor compound of the present technology may be integrated in the anode film 14, the solid electrolyte 16 or the cathode film 18.

In certain embodiments, the lithium salt included in the solid electrolyte 16 may be in LiCF₃SO₃, LiB (C₂O₄)₂, LiN(CF₃SO₂)₂, LiN(FSO₂)₂, LiC(CF₃SO₂)₃, LiC(CH₃)(CF₃SO₂)₂, LiCH (CF₃SO₂)₂, LiCH₂(CF₃SO₂), LiC₂F₅SO₃, LiN(C₂F₅SO₂)₂, LiN(CF₃SO₂), LiB(CF₃SO₂)₂, LiPF₆, LiSbF₆, LiClO₄, LiSCN, LiAsF₆, or LiBF₄.

The internal operating temperature of the battery 10 in the electrochemical cells 12 is typically between about 40° C. and about 100° C. Lithium polymer batteries preferably include an internal heating system to bring the electrochemical cells 12 to their optimal operating temperature. The battery 10 may be used indoors or outdoors in a wide temperature range (between about −40° C. to about +70° C.).

### EXAMPLES

The examples below are given to illustrate the practice of various embodiments of the present disclosure. They are not intended to limit or define the entire scope of this disclosure.

### Example 1: Synthesis of a Thiol Functionalized Single-Ion Conductor According to One Embodiment of the Present Technology

Briefly, 1,3,propanedithiol (2.12 g, 19.6 mmol, Sigma-Aldrich, >99%) was dissolved in 20 mL anhydrous THF (Sigma-Aldrich) in an oven-dried round bottom flask. The solution was purged with argon for ˜40 mins and then cool to 0° C. triethylamine (0.45 g, 4.53 mmol, Sigma-Aldrich, >99%) was dissolved in 2 mL anhydrous THF and added to the flask. Then lithium 1-[3-(acryloyloxy)-propylsulfonyl]-1-(trifluoromethylsulfonyl)imide (J503, 5.0 g, 15.1 mmol) was dissolved in 15 mL anhydrous THF and added to the flask dropwise. The reaction was stirred and warmed up to room temperature overnight (˜18 hrs). After the reaction, THF was evaporated and the crude product was precipitated into 200 mL hexane four times to remove excess dithiol. The viscous liquid product was collected and dried on an rotary evaporator. ˜3.7 g clear, light yellow viscous J517 was obtained (85% yield).

The synthetic route for the preparation of the thiol functionalized single-ion conductor according to this embodiment is represented below:

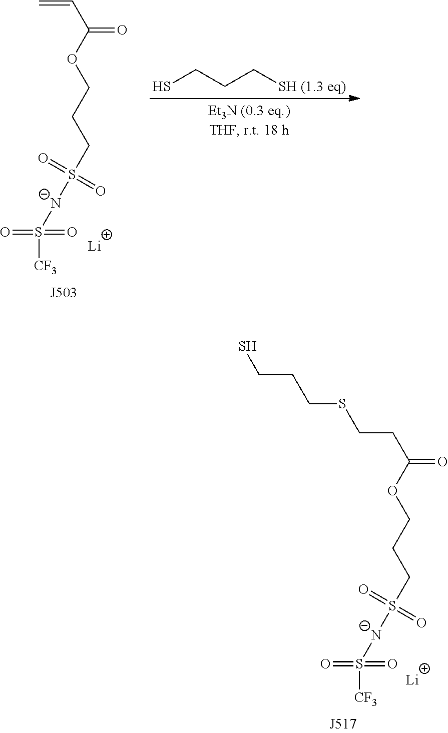

### Example 2: Synthesis of a Thiol Functionalized Single-Ion Conductor According to Another Embodiment of the Present Technology

1,3-propanedithiol (0.52 g, 4.8 mmol), lithium 1-[3-(acryloyloxy)-propylsulfonyl]-1-(trifluoromethylsulfonyl)imide (J503, 0.79 g, 2.4 mmol), photoinitiator 2,2-Dimethoxy-2-phenylacetophenone (DMPA, 8 mg, 0.03 mmol, Sigma-Aldrich, 99%) were dissolved in 5 mL anhydrous THF. The solution was purged with argon for 20 mins and irradiated with 365 nm UV lamp (VWR) for 30 mins under stirring at room temperature. After the reaction, THF was evaporated and the crude product was washed with hexane four times and then vac-dried at 100° C. for ˜2 hrs. Finally, 0.85 g clear light yellow liquid was obtained (80% yield).

The synthetic route for the preparation of the thiol functionalized single-ion conductor according to this embodiment is represented below:

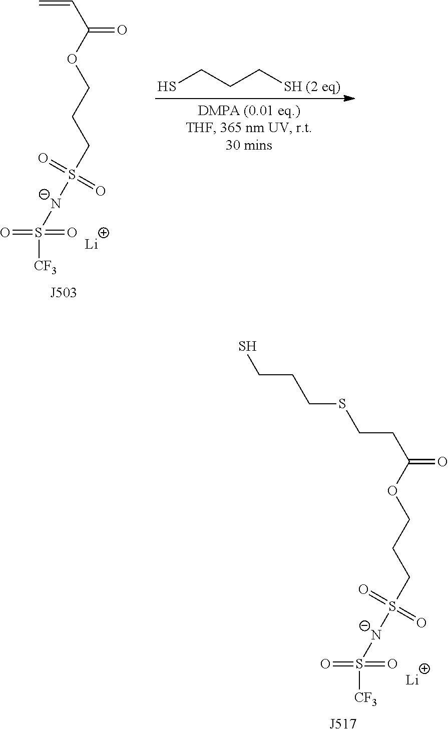

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the present technology is not limited to the particular embodiments described and illustrated herein but includes all modifications and variations falling within the scope of the present technology as defined in the appended claims.

All references cited in this specification, and their references, are incorporated by reference herein in their entirety where appropriate for teachings of additional or alternative details, features, and/or technical background.

What is claimed is:

1. A thiol functionalized conductor compound having formula I: 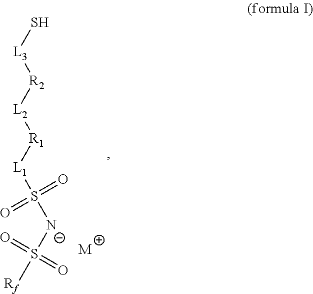
wherein:
R_{f} is F, CF₃, CF₂CF₃, (CF₂)ₙCF₃ wherein n is ≥1, C₆F₅, a branched C₃-C₄ fluoroalkyl group, —(CF₂CF₂O)ₘ—CF₂CF₃ wherein m=1, 2 or 3, —(CF₂O)ₚ/(CF₂CF₂O)_{q}—CF₂CF₃ wherein 1≤p≤10, 1≤q≤10, and the (CF₂O) and (CF₂CF₂O) units are randomly copolymerized, or an aryl substituted with at least one fluorine, and at least one electron-withdrawing group;
M⁺ is a monovalent cation;
L₁ is a n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
L₂ is absent or present and when present is a n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
L₃ is absent or present and when present is n-alkyl group, a fluorinated alkyl group, a branched alkyl group, an ethylene oxide linker, a fluorinated ethylene oxide linker, a cycloalkyl group, a fluorinated cycloalkyl group, a phenyl group, or a fluorinated phenyl group;
R₁ is an ether, a thioether, an ester, an amide, a urethane, urea, a secondary amine, or a tertiary amine; and
R₂ is absent or present and when present is an ether, a thioether, an ester, an amide, a urethane, urea, a secondary amine, or a tertiary amine.

2. The thiol functionalized conductor compound of claim 1, wherein R_{f} is CF₃.

3. The thiol functionalized conductor compound of claim 1, wherein the electron withdrawing group is —CN, —NO₂, —CF₃, or —SO₂CF₃.

4. The thiol functionalized conductor compound of claim 1, wherein the monovalent cation is an alkali metal cation.

5. The thiol functionalized conductor compound of claim 4, wherein the alkali metal cation is H⁺, K⁺, Na⁺, Li⁺, Rb⁺, or Cs⁺.

6. The thiol functionalized conductor compound of claim 4, wherein the alkali metal cation is Li⁺.

7. The thiol functionalized conductor compound of claim 1, wherein L₁ is a n-alkyl group and n is 1, 2, 3, 4, 5 or 6.

8. The thiol functionalized conductor compound of claim 7, wherein L₁ is (CH₂)₃.

9. The thiol functionalized conductor compound of claim 1, wherein L₂ is a n-alkyl group and n is 1, 2, 3, 4, 5 or 6.

10. The thiol functionalized conductor compound of claim 9, wherein L₂ is (CH₂)₂.

11. The thiol functionalized single ion conductor compound of claim 1, wherein L₃ is a n-alkyl group and n is 2, 3, 4, 5 or 6.

12. The thiol functionalized conductor compound of claim 11, wherein L₃ is (CH₂)₃.

13. The thiol functionalized conductor compound of claim 1, wherein R₁ is an ester.

14. The thiol functionalized conductor compound of claim 1, wherein R₂ is a thioether.

15. The thiol functionalized conductor compound of claim 1, having formula II or formula III:

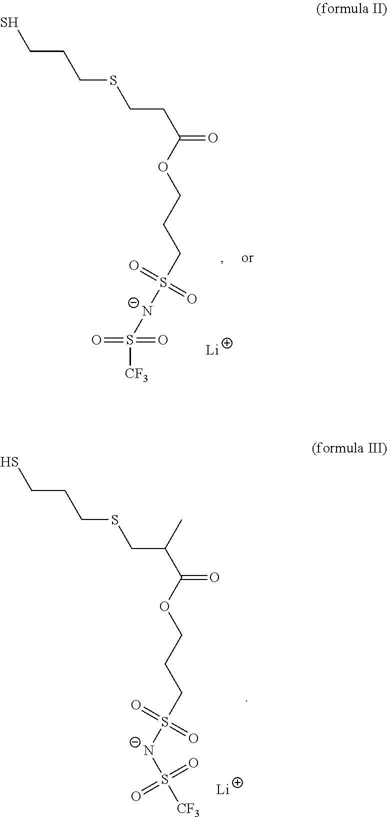 [SMILES: O=C(CCSCCCS)OCCCS(=O)(=O)[N-]S(=O)(=O)C(F)(F)F ; [Li+] ; CC(CSCCCS)C(=O)OCCCS(=O)(=O)[N-]S(=O)(=O)C(F)(F)F ; [Li+]]

16. A method for the synthesis of the thiol functionalized conductor compound of claim 1, the method comprising reacting a thiol compound with a lithium bis(trifluoromethanesulfonyl)imide (LiTFSI)-monomer having a C═C in its backbone.

17. The method of claim 16, wherein the LiTFSI-monomer compound has the following formula IV, formula V, formula VI, formula VII, or formula VIII:

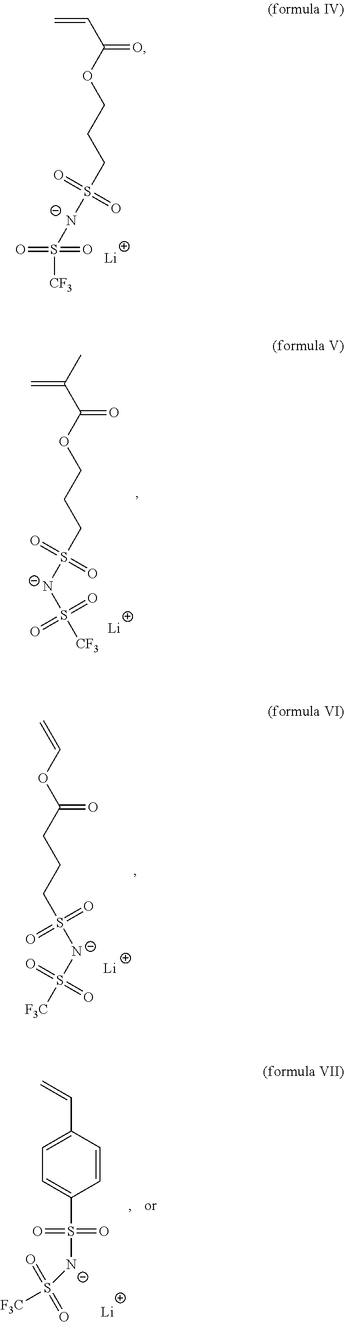

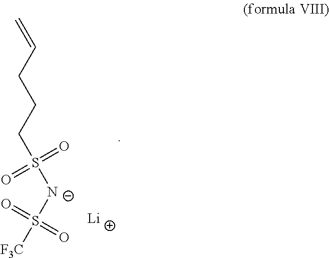 [SMILES: C=CCCCS(=O)(=O)[N-]S(=O)(=O)C(F)(F)F ; [Li+]]

18. The method of claim 16, wherein the thiol compound is 1,3-propane-dithiol or 2,2′-(Ethylenedioxy)diethanethiol.

19. A single-ion conducting polymer electrolyte, cathode or anode comprising a thiol functionalized conductor compound as defined in claim 1.

20. A solid-state battery comprising a positive electrode, a negative electrode and the single-ion conducting polymer electrolyte of claim 19.
